(19) United States
(12) Patent Application Publication
(10) Pub. No.: US 2025/0120622 A1
(43) Pub. Date: Apr. 17, 2025

(54) PATIENT CONNECTOR ASSEMBLY WITH VERTICAL DETENTS

(71) Applicant: MASIMO CORPORATION, Irvine, CA (US)

(72) Inventors: Stephen Scruggs, Newport Beach, CA (US); Yassir Kamel Abdul-Hafiz, Irvine, CA (US); Ammar Al-Ali, San Juan Capistrano, CA (US); Marc Antoine Laidet, Mission Viejo, CA (US)

(21) Appl. No.: 18/929,297

(22) Filed: Oct. 28, 2024

Related U.S. Application Data
(63) Continuation of application No. 17/815,689, filed on Jul. 28, 2022, now Pat. No. 12,156,732.
Application No. 17/815,689 is a continuation of application No. 16/597,503, filed on Oct. 9, 2019, now Pat. No. 11,445,948.
(60) Provisional application No. 62/744,456, filed on Oct. 11, 2018.

(51) Int. Cl. A61B 5/1455; A61B 5/00; A61B 5/024; A61M 16/08

(52) U.S. Cl. CPC A61B 5/14551; A61B 5/02444; A61B 5/6826; A61M 16/0816; A61B 2562/166; A61B 2562/227; A61B 2562/228

(57) ABSTRACT

The present disclosure includes a connector assembly that is a part of a sensor assembly for collecting patient physiological data. The connector assembly can include a first connector tab with catches, a second connector tab with openings, a retainer with pins, and a circuit board coupled to a cable. Each of the pins of the retainer can include a detent that can engage one of the catches of the first connector tab. The pins can extend through the openings of the second connector tab so that the retainer is coupled to the first connector tab by the detents engaging the catches. The first and second connector tabs can thereby be coupled together to support the circuit board and the cable between the first and second connector tabs.

### RELATED APPLICATIONS

This application is a continuation of U.S. patent application Ser. No. 17/815,689, filed Jul. 28, 2022, titled “PATIENT CONNECTOR ASSEMBLY WITH VERTICAL DETENTS”, which is a continuation of U.S. patent application Ser. No. 16/597,503, filed Oct. 9, 2019, issued as Ser. No. 11,445,948, titled “PATIENT CONNECTOR ASSEMBLY WITH VERTICAL DETENTS”, which claims priority benefit under 35 U.S.C. § 119 (e) from U.S. Provisional Application No. 62/744,456, filed Oct. 11, 2018, titled “PATIENT CONNECTOR ASSEMBLY WITH VERTICAL DETENTS”; the disclosure of each of which is hereby incorporated by reference in its entirety.

### BACKGROUND

### Field of Disclosure

The disclosure related to improving the efficiency of manufacturing connector assemblies for sensor assemblies that collect patient physiological data.

### Description of the Related Art

Pulse oximetry is a widely accepted noninvasive procedure for measuring the oxygen saturation level of arterial blood, an indicator of a person's oxygen supply. A typical pulse oximetry system utilizes an optical sensor clipped onto a fingertip to measure the relative volume of oxygenated hemoglobin in pulsatile arterial blood flowing within the fingertip. Oxygen saturation (SpO₂), pulse rate, and a plethysmograph waveform, which is a visualization of pulsatile blood flow over time, are displayed on a monitor accordingly.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight), and electrosurgical instrument interference, which are scenarios in which conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. No. 7,647,083, filed Mar. 1, 2006, titled Multiple Wavelength Sensor Equalization; U.S. patent application Ser. No. 11/367,036, filed Mar. 1, 2006, titled Configurable Physiological Measurement System; U.S. patent application Ser. No. 11/367,034, filed Mar. 1, 2006, titled Physiological Parameter Confidence Measure and U.S. patent application Ser. No. 11/366,208, filed Mar. 1, 2006, titled Noninvasive Multi-Parameter Patient Monitor, all assigned to Cercacor Laboratories, Irvine, CA (Cercacor) and all incorporated by reference herein. Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY

The present disclosure describes a connector assembly including a bottom connector tab, a top connector tab, and a retainer with pins having detents. The bottom tab of the connector assembly can include catches that can mate with the detents of the retainer that extend through the top connector tab to provide sufficient force to hold the bottom and top connector tabs together. The connector assembly can be part of a cable or a sensor of a patient monitoring system and used for gathering and transmitting real-time patient data to one or more monitors or other devices. The connector assembly can include a cable assembly with a circuit board coupled to one end of the cable assembly.

The retainer and the bottom and top connector tabs can together provide an improved overall construction of the connector assembly. Connector assemblies can often be relatively small, and the manufacturing of connector assemblies using fasteners, such as screws, that may utilize tools, such as screwdrivers, can be difficult. The retainer and connector tabs disclosed herein, however, can be assembled simply by pushing the retainer downward to fixedly mate the bottom and top connector tabs, so the manufacturing process can be easy and quick.

In some embodiments, a noninvasive physiological sensor assembly is disclosed. The noninvasive physiological sensor assembly can monitor a tissue of a patient. The noninvasive physiological sensor assembly can include a noninvasive sensor, a cable assembly, and a connector assembly. The cable assembly can include a circuit board and conductors. The circuit board can be coupled to a proximal end of the conductors. The cable assembly can be coupled to the noninvasive sensor at a distal end of the conductors. The connector assembly can include a first connector tab, a second connector tab, and a retainer. The first connector tab can include a plurality of catches. The second connector tab can include a plurality of openings. The retainer can include a plurality of pins having a plurality of detents. The plurality of pins can extend through the plurality of openings so that the plurality of detents engage the plurality of catches to support the circuit board and the cable assembly between the first connector tab and the second connector tab.

The noninvasive physiological sensor assembly of the preceding paragraph can include one or more of the following features: The retainer can include a base. The base can be positioned in an inset portion of the second connector tab when the plurality of detents engage the plurality of catches. The noninvasive sensor can include a pulse oximeter. A portion of the circuit board and a portion of the conductors can be positioned between the first connector tab and the second connector tab. The first connector tab can include a first channel and the second connector tab can include a second channel. The first channel and the second channel can together define an aperture of the connector assembly. The conductors and the circuit board can be positioned between the first connector tab and the second connector tab so that at least a portion of the conductors is positioned within the aperture. The plurality of openings extend through the second connector tab. The plurality of catches can be disposed on an inner surface of the first connector tab and extend upward and away from the inner surface. The plurality of openings can be associated with the plurality of catches and positioned above the plurality of catches. The plurality of openings and the plurality of catches can be vertically aligned. The plurality of openings and the plurality of catches can be configured to align and couple the first connector tab and the second connector tab.

A method of manufacturing the noninvasive physiological sensor assembly of the preceding two paragraphs is additionally disclosed.

In some embodiments, a cable assembly for a noninvasive sensor assembly is disclosed. The cable assembly can monitor a tissue of a patient. The cable assembly can include conductors, a circuit board, and a connector assembly. The circuit board can be coupled to a proximal end of the conductors. The connector assembly can include a first connector tab, a second connector tab, and a retainer. The first connector tab can include a plurality of catches. The second connector tab can include a plurality of openings. The retainer can include a plurality of pins having a plurality of detents. The plurality of pins can be dimensioned to extend through the plurality of openings so that the plurality of detents engage the plurality of catches to support the circuit board and the conductors between the first connector tab and the second connector tab.

The cable assembly of the preceding paragraph can include one or more of the following features: The retainer can include a base. The base can be positioned in an inset portion of the second connector tab when the plurality of detents engage the plurality of catches. A portion of the circuit board and a portion of the conductors can be positioned between the first connector tab and the second connector tab. At least a portion of the conductors can extend through a first cutout of the first connector tab and at least a portion of the circuit board can extend through a second cutout of the first connector tab. The plurality of openings can include grooves configured to push the detents downwards towards the first connector tab to cause the pins and the retainer to be pushed downwards towards the first connector tab. The pins can be aligned with the plurality of catches so that at least a portion of the plurality of detents can engage top portions of the plurality of catches.

A method of manufacturing the cable assembly of preceding two paragraphs is additionally disclosed.

In some embodiments, a method of manufacturing a connector assembly for a noninvasive sensor assembly is disclosed. The method can include: placing a circuit board and a portion of conductors on an inner surface of a first connector tab having a plurality of catches; placing a second connector tab on top of the first connector tab so that an inner surface of the second connector tab can face the inner surface of the first connector tab, the second connector tab having a plurality of openings; and extending a retainer that can include a plurality of pins having a plurality of detents through the plurality of openings so that the plurality of detents can engage the plurality of catches.

The method of the preceding paragraph can include one or more of the following features: The method can further include applying an adhesive between the circuit board and the inner surface of the first connector tab. The method can further include aligning the second connector tab with the first connector tab to align the plurality of openings with the plurality of catches. The method can further include: extending the retainer a first distance through the plurality of openings to cause the plurality of pins to bend away from the plurality of catches; and extending the retainer a second distance through the plurality of openings to cause the plurality of pins to snap back towards the plurality of catches and engage with top portions of the plurality of catches.

For purposes of summarizing the disclosure, certain aspects, advantages and novel features are discussed herein. It is to be understood that not necessarily all such aspects, advantages or features will be embodied in any particular embodiment and an artisan would recognize from the disclosure herein a myriad of combinations of such aspects, advantages or features.

### BRIEF DESCRIPTION OF THE DRAWINGS

FIG. 1A illustrates a top view of a sensor assembly.

FIG. 1B illustrates an exploded perspective view of the sensor assembly of FIG. 1A.

FIG. 2A illustrates a perspective view of a sensor assembly.

FIG. 2B illustrates an exploded perspective view of the sensor assembly of FIG. 2A.

FIG. 3A illustrates a top view of a connector assembly and a cable assembly of the sensor assembly of FIGS. 1A, 1B, 2A, and 2B.

FIG. 3B illustrates a schematic diagram of the sensor assembly of FIGS. 1A and 1B.

FIG. 4A illustrates a top view of a bottom connector tab of the patient connector assembly of FIGS. 1A, 1B, 2A, and 2B.

FIG. 4B illustrates a perspective view of the bottom connector tab of FIG. 4A.

FIG. 4C illustrates a bottom view of a cable assembly.

FIG. 4D illustrates a perspective view of the cable assembly of FIG. 4C and

the bottom connector tab of FIG. 4B.

FIG. 4E illustrates a top view of the cable assembly of FIG. 4D positioned on the bottom connector tab of FIG. 4B.

FIG. 4F illustrates a perspective, exploded view of the patient connector assembly of FIG. 1A including the bottom connector tab of FIG. 4B and the cable assembly of FIG. 4D.

FIG. 4G illustrates a perspective view of the patient connector assembly of FIG. 4F.

### DETAILED DESCRIPTION

A connector assembly is disclosed that can be part of a sensor assembly usable to collect patient physiological data. The connector assembly can include first and second connector tabs, and a circuit board of a cable assembly for the sensor assembly placed between the first connector tab and the second connector tab. The connector assembly can further include a retainer that can extend through the second connector tab and be coupled to the first connector tab to secure the first and second connector tabs together. The sensor assembly can include a noninvasive sensor that may be positioned proximate to a patient's skin for generating the patient physiological data.

The first connector tab can include catches disposed on an inner surface of the first connector tab. The second connector tab can include openings, where the openings correspond to the catches. The retainer can include pins, where each of the pins correspond to the openings and the catches of the second and first connector tabs, respectively.

The pins of the retainer can extend through the openings of the second connector assembly. The detents of the pins can engage the catches of the first connector tab when the retainer is pushed down towards the first connector tab. The engagement between the catches and the detents of the pins of the retainer can cause the first connector tab and the second connector tab to align and mate, thereby securing the circuit board between the first connector tab and the second connector tab.

To facilitate a complete understanding of the disclosure, the remainder of the detailed description references the drawings, wherein like references number are references with numerals throughout.

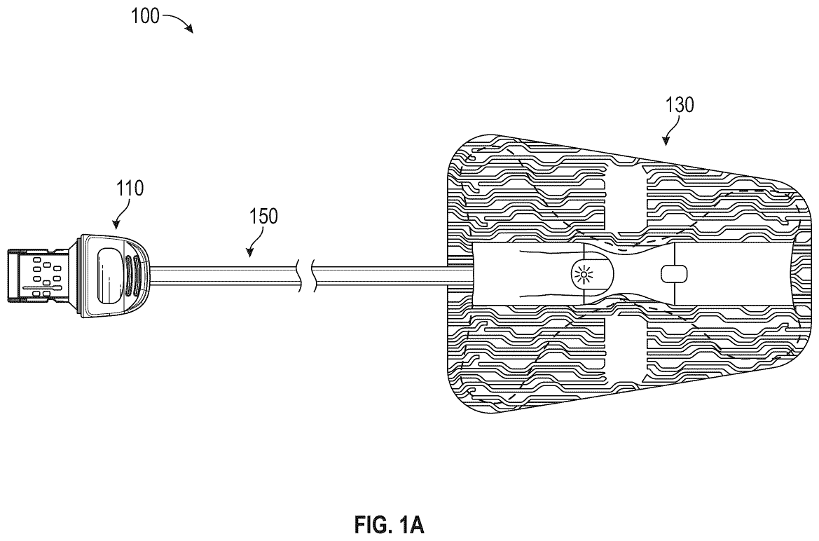

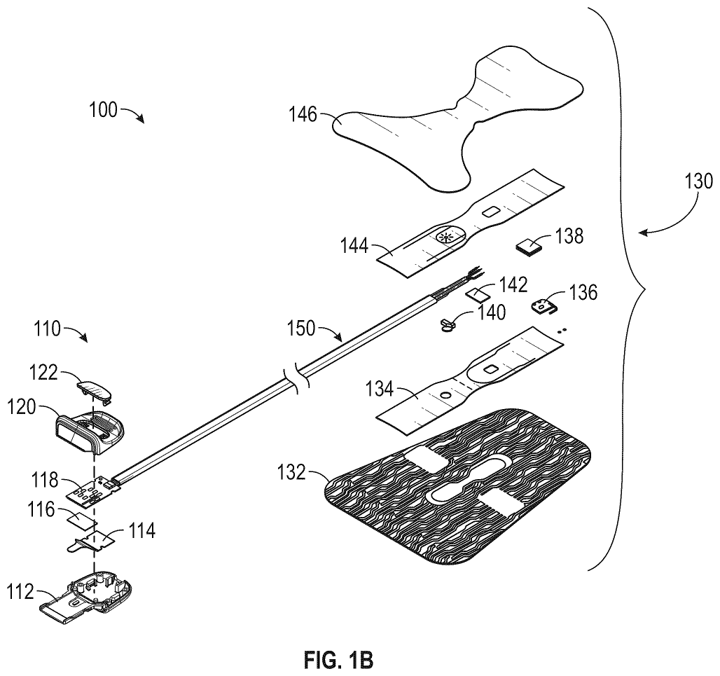

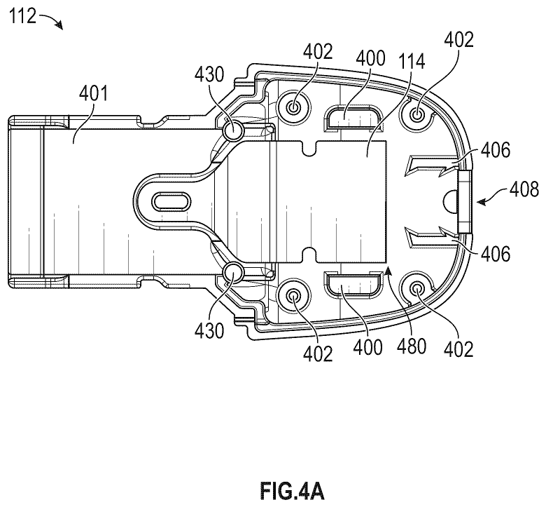

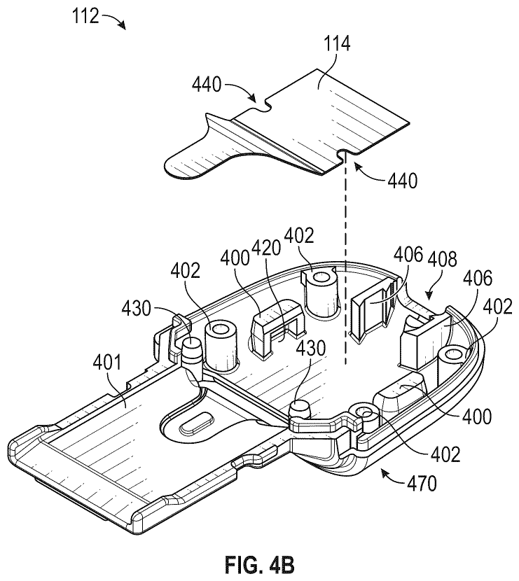

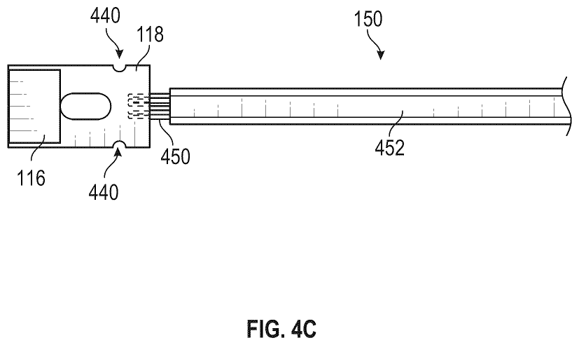

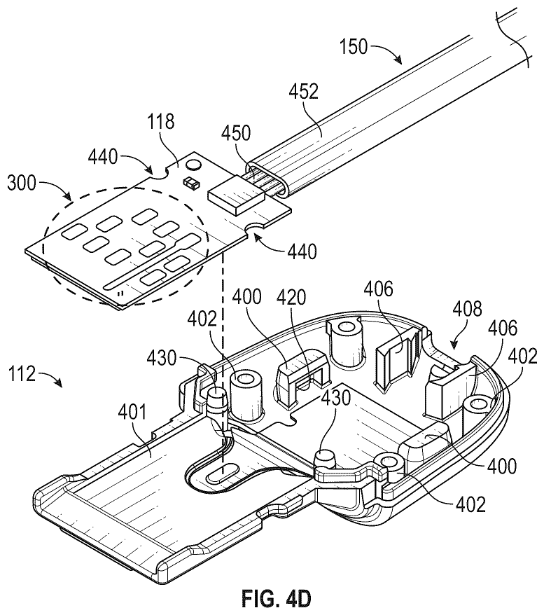

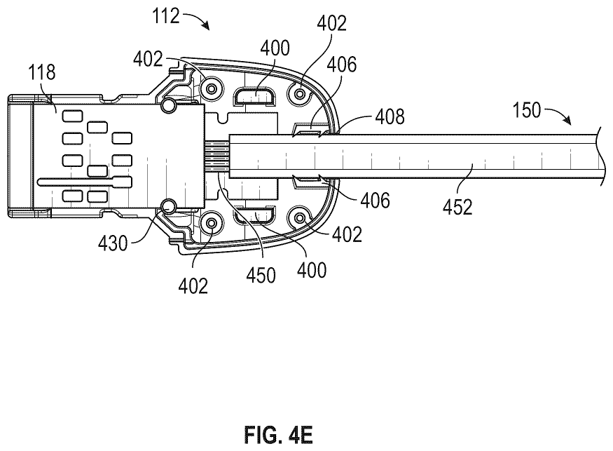

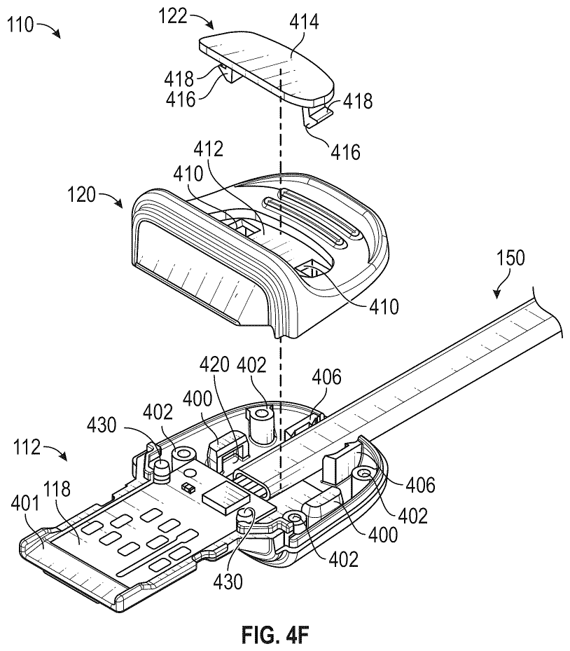

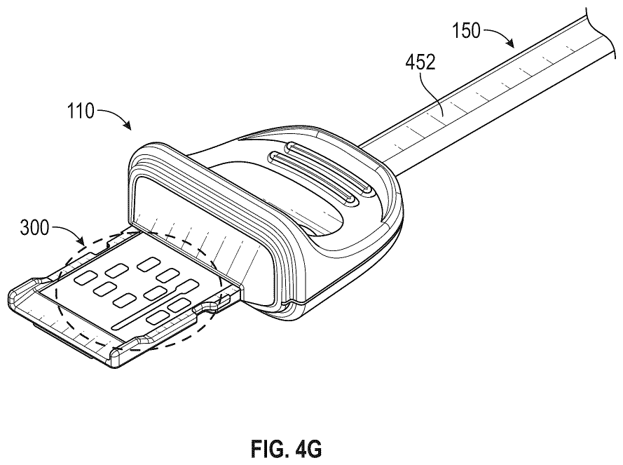

FIG. 1A illustrates a sensor assembly 100 including a connector assembly 110 coupled to a sensor 130 via a cable assembly 150. The cable assembly 150 can include a round or flat cable 452 with conductor cords 450 (shown in FIGS. 4C-4F) that can transmit digital or analog signals between the sensor 130 and the connector assembly 110. The sensor 130 may be a noninvasive oximetry sensor that can be positioned proximate to or adhered to a patient's body. Examples of the connector assembly 110 are disclosed in U.S. Pat. No. 9,697,928, U.S. Patent Pub. No. 2016/0234944, and U.S. Patent Pub. No. 2016/0233632, which are incorporated by reference herein.

FIG. 1B shows an exploded, perspective view of the sensor assembly 100 shown in FIG. 1A. The connector assembly 110 can include a first connector tab 112, a connector shield 114, an adhesive 116, a circuit board 118, a second connector tab 120, and a retainer 122. The circuit board 118 can be part of the cable assembly 150. The sensor 130 can include a liner 132, a first tape 134, a detector 136, a detector shield 138, an emitter 140, an adhesive 142, a second tape 144, and an applicator 146. The sensor 130 can position the detector 136 and the emitter 140 proximate to a tissue of a patient for monitoring.

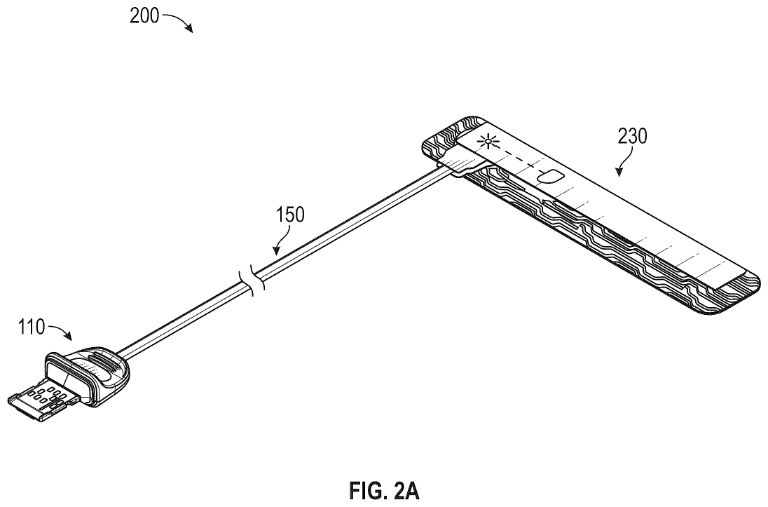

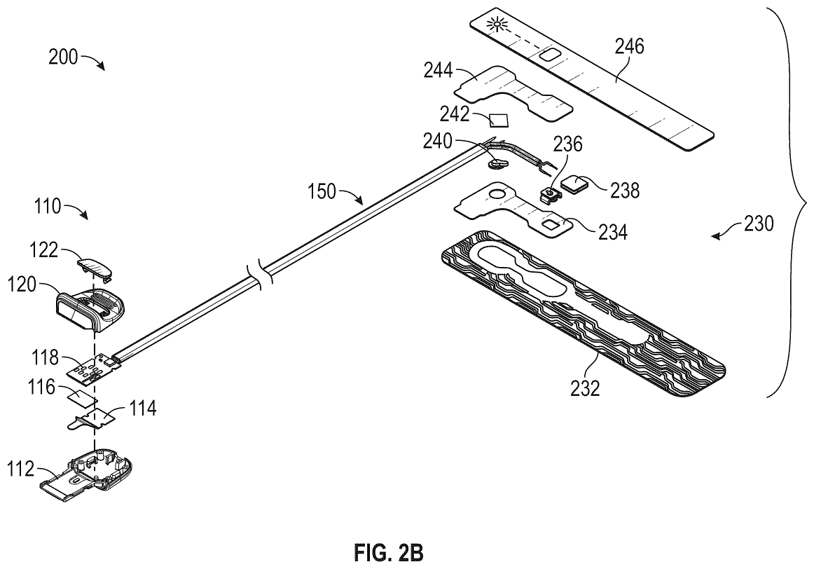

FIG. 2A shows a perspective view of a sensor assembly 200 including the connector assembly 110 coupled to a sensor 230 via the cable assembly 150. The sensor 230 can be designed for use by neonates (for example, humans less than four months old) while the sensor 130 can be designed for individuals older than neonates.

FIG. 2B illustrates an exploded perspective view of the sensor assembly 200 shown in FIG. 2A. The sensor 230 can include a liner 232, a first tape 234, a detector 236, a detector shield 238, an emitter 240, an adhesive 242, a second tape 244, and an applicator 246. The sensor 230 can position the detector 236 and the emitter 240 proximate to a tissue of a patient for monitoring.

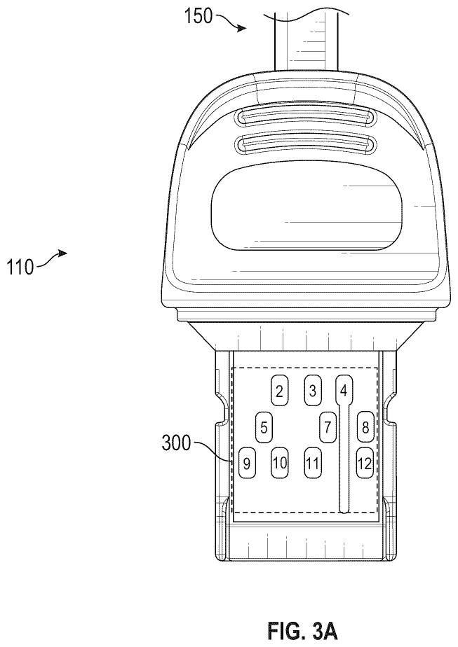

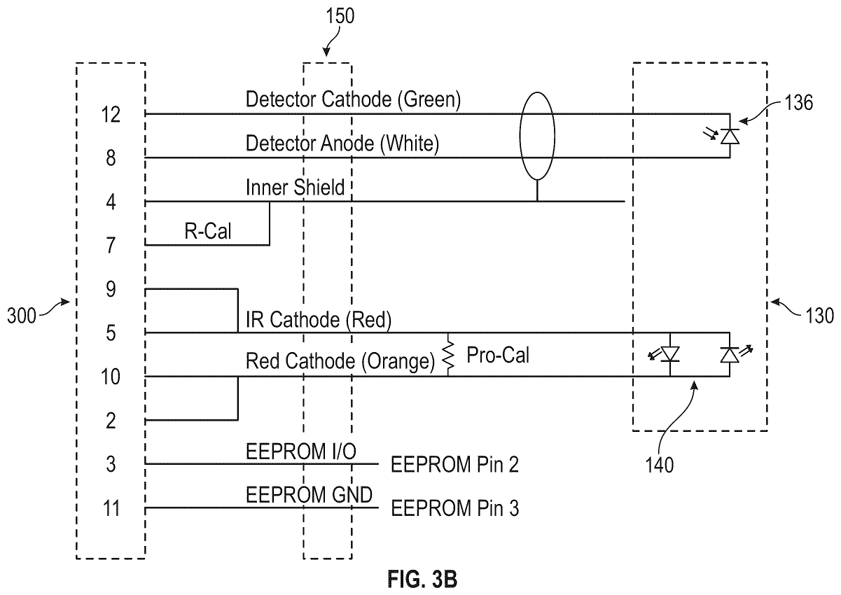

FIG. 3A shows a top view of the connector assembly 110 and the cable assembly 150.

The connector assembly 110 can include electrical contacts 300, which are numbered 2, 3, 4, 5, 7, 8, 9, 10, 11, and 12 in FIGS. 3A and 3B for reference. The electrical contacts 300 can be on the circuit board 118. The number of electrical contacts 300 or the size of the electrical contacts 300 on the circuit board 118 can vary from the illustration in FIG. 3A and depend on a number of signals transmitted or types of signals transmitted. The electrical contacts 300 can be printed on the circuit board 118. The size of the connector assembly 110 can vary depending on the number or size of electrical contacts 300. The shapes or locations of each of the electrical contacts 300 on the circuit board 118 can vary from the illustration in FIG. 3A and depend on a number of signals transmitted or types of signals transmitted.

FIG. 3B shows a schematic diagram of the sensor assembly 100 with the electrical contacts 300. The electrical contacts 300 can facilitate electrical communication between a patient monitor and a memory (for example, EEPROM), and the detector 136 and the emitter 140 of the sensor 130. The electrical contacts 300 can also include one or more contacts that serve as an electrical ground.

FIG. 4A shows a top view of the first connector tab 112 of the connector assembly 110 with the connector shield 114. The first connector tab 112 can include a platform 401, catches 400, receptors 402, guides 406, a cable cutout 408, and protrusions 430.

The first connector tab 112 can have an outer surface 470 and an inner surface 480. The catches 400, the receptors 402, and the guides 406 can be disposed on the inner surface 480. The catches 400, the receptors 402, and the guides 406 can extend upward and away from the inner surface 480. Additionally or alternatively, the catches 400, the receptors 402, and the guides 406 can extend in a direction that is substantially orthogonal to a plane defined by the inner surface 480.

The guides 406 can form a channel for aligning the cable assembly 150 within the first connector tab 112. The distance between the guides 406 can be greater than or equal to a width of the cable 452 of the cable assembly 150. The guides 406 can have a length that can be greater than or equal to, or less than or equal to the width of the cable 452 of the cable assembly 150.

The cable cutout 408 can be dimensioned to receive the cable 452 of the cable assembly 150. The cable cutout 408 can be characterized by a width and a height, where the width and the height can be sufficient to allow at least a portion of the cable assembly 150 to be placed within the cable cutout 408. The second connector tab 120 can have a cable cutout that is similar to the cable cutout 408. The cable cutout of the second connector tab 120 may allow at least a portion of the cable assembly 150 to be similarly placed within the second connector tab 120. The cable cutout 408 and the cable cutout of the second connector tab 120 can together form an aperture sized to receive the cable 452 of the cable assembly 150.

The protrusions 430 can extend from the inner surface 480. The protrusions 430 can act as a guide for notches 440 (see FIG. 4C) of the circuit board 118. The protrusions 430 can be circular, as shown in FIGS. 4A and 4B, or rectangular, triangular, among other possible shapes, to match a shape of the notches 440.

The first connector tab 112 can include a cutout dimensioned to receive at least a portion of the circuit board 118. The second connector tab 120 can similarly include a cutout dimensioned to receive at least a portion of the circuit board 118. The circuit board cutouts of the first connector tab 112 and the second connector tab 120 can together form a circuit board aperture sized to receive the circuit board 118. The circuit board aperture can be positioned opposite from the aperture configured to receive the cable 452. The circuit board aperture can be positioned and extend between the protrusions 430.

The connector shield 114 can be adhered to the inner surface 480 of the first connector tab 112. The size and dimension of the connector shield 114 can correspond to the size of the circuit board 118, the size of the first connector tab 108, or the size of the connector assembly 110.

FIG. 4B shows an exploded perspective view of the first connector tab 112 with the connector shield 114 positioned for placement on the first connector tab 112. As shown in FIG. 4B, the catches 400, the receptors 402, the guides 406, and the protrusions 430 can extend from the inner surface 480 of the first connector tab 112. The catches 400 each can include a groove 420. The grooves 420 of the catches 400 can face each other as illustrated in FIG. 4B or may not face each other (for example, may face away from each other). The grooves 420 of the catches 400 can each be a space between the inner surface 480 of the first connector tab 112, an end of one of the catches 400 opposite the inner surface 480 of the first connector tab 112, and a side of one of the catches 400.

FIG. 4C illustrates a bottom view of the cable assembly 150 with the circuit board 118. The cable assembly 150 can include conductor cords 450 that encase conductors (for example, wires) that carry signals between the sensor 130 and the circuit board 118. The conductors can be coupled to the circuit board 118, thus creating an electrical connection between the conductors and the circuit board 118. The conductors can be coupled to either a top surface or a bottom surface of the circuit board 118. The conductor cords 450 can, in turn, be encased at least partly by the cable 452 that provides support and groups the conductor cords 450 as part of a single cable.

As previously described, the circuit board 118 can include notches 440. The notches 440 can be disposed on the side of the circuit board 118 as shown in FIG. 4C. The notches 440 can engage the protrusions 430 of the first connector tab 112 to retain the circuit board 118 to the first connector tab 112. The notches 440 can be circular, as shown in FIG. 4C, rectangular, triangular, among other shapes. The shape of the notches 440 can correspond to that of the protrusions 430 to enable the notches 440 and the protrusions 430 to engage one another. The notches 440 and the protrusions 430 can together hold the circuit board 118 in place and prevent longitudinal movement (which can be in a direction along a length of the connector assembly 110) and horizontal movement (which can be in a direction along a width of the connector assembly 110) of the circuit board 118 with respect to the first connector tab 112 and the second connector tab 120.

The circuit board 118 can be adhered to the inner surface of the first connector tab 112 via the adhesive 116. The adhesive 116 can be applied to a bottom surface of the circuit board 118 so that it does not interfere with transmission of signals via the conductors. The adhesive 116 can additionally or alternatively be placed (i) between the bottom surface of the circuit board 118 and the inner surface 480 of the first connector tab 112 or (ii) between the bottom surface of the circuit board 118 and the platform 401 of the first connector tab 112.

FIG. 4D illustrates a perspective view of the cable assembly 150 and the first connector tab 112 with the cable assembly 150 positioned for placement on the first connector tab 112. The circuit board 118 can be positioned on top of the first connector tab 112 so that the electrical contacts 300 face away from the platform 401, thereby allowing the electrical contacts 300 be exposed after the second connector tab 120 is positioned on top of the first connector tab 112.

As shown in FIG. 4E, the circuit board 118 can be laid on top of the platform 401 of the first connector tab 112, while the cable 452 of the cable assembly 150 can be laid on top of the cable cutout 408 (see FIG. 4B). The platform 401 can provide support for the circuit board 118 when the circuit board 118 is placed on the first connector tab 112. As discussed herein, the circuit board 118 can be adhered to the platform 401 using the adhesive 116. The protrusions 430 can correspond to the plurality of notches 440 of the circuit board 118 as discussed above. The protrusions 430 and the notches 440 can assist with aligning or securing the circuit board 118 to the first connector tab 112 and may prevent the circuit board 118 from moving along an axis defined by a length or a width of the circuit board 118.

The guides 406 can be located proximate to the cable cutout 408 to prevent the cable 452 of the cable assembly 150 from bending near the cable cutout 408, thereby preventing damage to the cable assembly 150. The guides 406 can prevent the cable assembly 150 from moving along a length or a width of the cable assembly 150.

FIG. 4F illustrates an exploded perspective view of the connector assembly 110 and the cable assembly 150. As can be seen, the second connector tab 120 is positioned above of the first connector tab 112 and the cable assembly 150. The second connector tab 120 and the first connector tab 112 can be brought together to secure at least a portion of the cable assembly 150 and the circuit board 118 between the second connector tab 120 and the first connector tab 112. The second connector tab 120 can include pins configured to engage the receptors 402 of the first connector tab 112. The pins and the plurality of receptors 402 can align the first connector tab 112 and the second connector tab 120 with respect to one another for coupling and assist with securing the first connector tab 112 and the second connector tab 120 together.

When the first connector tab 112 and the second connector tab 120 are aligned, openings 410 of the second connector tab 120 and the catches 400 of the first connector tab 112 can also be aligned. Once the second connector tab 120 is placed on top of the first connector tab 112 and properly aligned, the retainer 122 can couple the first connector tab 112 and the second connector tab 120 to one another so that the inner surface of the second connector tab 120 and the inner surface of the first connector tab 112 face one another. The retainer 122 can prevent longitudinal movement (which can be along the length of the connector assembly 110) or horizontal movement (which can be along the width of the connector assembly 110) of the first connector tab 112 with respect to the second connector tab 120, or longitudinal movement or horizontal movement of the second connector tab 120 with respect to the first connector tab 112. The second connector tab 120 may not cover at least a portion of the platform 401 of the first connector tab 112 and the circuit board 118. The second connector tab 120 may not cover any of the electrical contacts 300 of the circuit board 118.

The second connector tab 120 can include an inset portion 412. The openings 410 can be formed on the inset portion 412. The inset portion 412 can be sufficiently inset to match a thickness of the retainer 122 and so that the retainer fills the inset portion 412 and the outer surface of the second connector tab 120 is substantially flush with an outer surface of the retainer 122 when the retainer 122 is inserted. The inset portion 412 can have a depth that is less than or equal to, or greater than or equal to the thickness of the retainer 122. The outer surface of the second connector tab 120 may not be flush with the outer surface of the retainer 122 in other implementations.

As shown in FIG. 4F, the retainer 122 can include a base 414 and a pins 416. The base 414 can have a shape that corresponds to the inset portion 412. The surface area of the inset portion 412 may be greater than a surface area of the base 414. Each of the pins 416 can include a detent 418. The detents 418 can engage the grooves 420 of the catches 400. The pins 416 can have a length so that the pins 416 can extend through the second connector tab 120 towards the inner surface 480 of the first connector tab 112 when the second connector tab 120 and the first connector tab 112 are coupled. The base 414 of the retainer 122 can have a shape that substantially matches a shape of the inset portion 412. For example, the base 414 and the inset portion 412 can both be substantially rectangular in shape so that the inset portion 412 can receive the base 414.

During manufacture, the retainer 122 can be moved towards the second connector tab 120 so that the pins 416 extend through the openings 410. The retainer 122 can then further be pushed towards the first connector tab 112 so that the pins 416 extend through the second connector tab 120. The detents 418 can engage the catches 400 to secure the first connector tab 112 to the second connector tab 120. The grooves 420 of the catches 400 can receive the detents 418 to prevent longitudinal movement (which can be along the length of the connector assembly 110), horizontal movement (which can be along the width of the connector assembly 110), or vertical movement (which can be away or toward the connector assembly 110) of the second connector tab 120 with respect to the first connector tab 112. The first connector tab 112 to the second connector tab 120 can together in turn support and secure the connector assembly 110 including the circuit board 118 and a portion of the cable assembly 150.

The pins 416 can generally align with the catches 400. When the retainer 122 is pushed down so that the pins 416 extend through the openings 410, the detents 418 can engage top portions of the catches 400. Pushing the retainer 122 further down towards the first connector tab 112 can cause the pins 416 to bend away from the catches 400 while the detents 418 engage the top portion of the catches 400.

The retainer 122 can be pushed down or extend through the openings 410 a first distance to cause the detents 418 to engage top portions of the catches 400. The retainer 122 can be pushed downward or extend through the openings 410 a second distance to cause the pins 416 to be pushed away from the catches 400. As discussed herein, continued engagement between the detents 418 and the top portions of the catches 400 can cause the pins 416 to bend away from the catches 400. Pushing down the retainer 122 further or extending the retainer 122 further through the openings 410 can cause the detents 418 to disengage from the catches 400. Disengagement between the detents 418 and the catches 400 can cause the pins 416 to snap back towards the catches 400. As the pins 416 snap back towards the catches 400, the detents 418 can engage the grooves 420 of the catches 400.

When the detents 418 no longer engage the top portion of the catches 400, the pins 416 can snap back towards the catches 400 to allow the detents 418 to engage the grooves 420 of the catches 400. The grooves 420 can engage the detents 418 to fix the pins 416 and thereby hold the retainer 122 in place. The engagement between the grooves 420 and the detents 418 can keep the first connector tab 112 and the second connector tab 120 mated. The engagement between the grooves 420 and the detents 418 can keep the first connector tab 112 and the second connector tab 120 aligned or coupled.

While the grooves 420 and the detents 418 are engaged, the grooves 420 can push the detents 418 downward towards the first connector tab 112, which in turn causes the pins 416 and the retainer 122 be pushed downward towards the first connector tab 112. Furthermore, the contact between the base 414 of the retainer 122 and the inset portion 412 of the second connector tab 120 can cause the base 414 to push the second connector tab 120 towards the first connector tab 112.

The retainer 122 can be integrated with the second connector tab 120 rather than separate from the second connector tab 120. The second connector tab 120 can include the pins 416 protruding towards the first connector tab 112 when the second connector tab 120 is placed on top of the first connector tab 112.

The retainer 122 can include one, two, three, four, or more of the pins 416. For example, the second connector tab 120 and the first connector tab 112 can have two openings and two catches, respectively, that correspond to two retainers 122 each with one of the pins 416.

The retainer 122 can be made of the same material as or different material from the second connector tab 120 and the first connector tab 112. The retainer 122 can be made out of plastic. The pins 416 can be made of the same material as or different material from the base 414. The pins 416 can include a flexible portion located between the pins 416 and the base 414. The flexible portion can act as a hinge to facilitate movement of the pins 416 when the pins 416 are pushed towards the grooves 420 of the first connector tab 112.

The second connector tab 120 can include one, two, three, or more sets of the openings 410 for the retainers 122. For example, the second connector tab 120 can include two sets of the openings 410 for two of the retainers 122 (for example, two separate retainers) in an example other than the example illustrated in FIG. 4F. In another example, the second connector tab 120 can include three or more sets of the openings 410 for three or more of the retainers 122.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements or states. Thus, such conditional language is not generally intended to imply that features, elements or states are in any way required for one or more embodiments or that one or more embodiments necessarily include logic for deciding, with or without author input or prompting, whether these features, elements or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Additionally, other combinations, omissions, substitutions and modifications will be apparent to the skilled artisan in view of the disclosure herein. Accordingly, the present disclosure is not intended to be limited by the reaction of the preferred embodiments, but is to be defined by reference to the appended claims.

Additionally, all publications, patents, and patent applications mentioned in this specification are herein incorporated by reference to the same extent as if each individual publication, patent, or patent application was specifically and individually indicated to be incorporated by reference.

1. (canceled)

2. A physiological sensor assembly comprising:
a physiological sensor;
a cable coupled to a circuit board at a distal end, wherein the cable is coupled to the physiological sensor at a proximal end;
a connector assembly comprising:
a first tab comprising:
a platform supporting the circuit board; and
a catch, wherein the platform and the catch are positioned on a first surface of the first tab;
a second tab comprising an opening, the second tab enclosing at least a portion of the first surface; and
a retainer comprising an arm further comprising a detent, the arm extending through the opening of the second tab and the detent engaging the catch of the first tab.

3. The physiological sensor assembly of claim 2, wherein the first tab comprises a guide contacting the cable, wherein the guide is positioned on the first surface of the first tab.

4. The physiological sensor assembly of claim 2, wherein the cable comprises a plurality of conductor cords, wherein a first portion of the conductor cords are encased by the cable, and wherein a second portion of the conductor cords proximate the circuit board are free of encasing by the cable.

5. The physiological sensor assembly of claim 3, wherein the second portion resides between the first tab and the second tab.

6. The physiological sensor assembly of claim 2, wherein the retainer comprises a base, the base being positioned in an inset portion of the second tab.

7. The physiological sensor assembly of claim 2, wherein the physiological sensor comprises an emitter and a detector.

8. The physiological sensor assembly of claim 2, wherein the circuit board comprises a notch configured to engage a protrusion of the first tab.

9. The cable assembly of claim 2, wherein at least a portion of the circuit board resides between the first tab and the second tab.

10. A cable assembly comprising:
a cable having a distal end coupled to a circuit board, the cable comprising a plurality of conductor cords, wherein a first portion of the conductor cords are encased by the cable, wherein a second portion of the conductor cords proximate the circuit board are free of encasing by the cable;
a connector assembly comprising:
a first tab comprising a catch;
a second tab comprising an opening; and
a retainer comprising an arm, the arm extending through the opening and comprising a detent, the detent engaging the catch,
wherein the second portion of the conductor cords resides between the first tab and the second tab.

11. The cable assembly of claim 10, wherein the first tab comprises a guide contacting the cable.

12. The cable assembly of claim 10, wherein the second portion of the conductor cords is enclosed by the first tab and the second tab.

13. The cable assembly of claim 10, wherein the retainer comprises a base, the base being positioned in an inset portion of the second tab.

14. The cable assembly of claim 10, wherein the engagement of the detent with the catch of the first tab aligns the first tab and the second tab.

15. The cable assembly of claim 10, wherein the circuit board comprises a notch configured to engage a protrusion of the first tab.

16. The cable assembly of claim 10, wherein the first tab comprises a first channel and the second tab comprises a second channel, the first channel and the second channel together defining an aperture.

17. The cable assembly of claim 16, wherein the circuit board is positioned between the first connector tab and the second connector tab so that at least a portion of the conductor cords are positioned within the aperture.

18. The cable assembly of claim 10, wherein the second tab comprises a plurality of openings through which a plurality of arms of the retainer extend.

19. The cable assembly of claim 18, wherein the plurality of arms comprises two arms.

20. The cable assembly of claim 10, wherein at least a portion of the circuit board extends through a cutout of the first connector tab.

21. The cable assembly of claim 10, wherein at least a portion of the circuit board resides between the first tab and the second tab.
